(19) United States
(12) Patent Application Publication
(10) Pub. No.: US 2015/0224212 A1
(43) Pub. Date: Aug. 13, 2015

(54) FERRITIN-BASED TUMOR TARGETING AGENT, AND IMAGING AND TREATMENT METHODS

(71) Applicants: Brown University, Providence, RI (US); Rhode Island Hospital, Providence, RI (US)

(72) Inventors: William Keun Chan Park, Westerly, RI (US); David R. Mills, Warwick, RI (US); Edward G. Walsh, Danielson, CT (US)

(21) Appl. No.: 14/626,071

(22) Filed: Feb. 19, 2015

Related U.S. Application Data
(63) Continuation of application No. PCT/US2013/055955, filed on Aug. 21, 2013.
(60) Provisional application No. 61/803,955, filed on Mar. 21, 2013.
(60) Provisional application No. 61/691,346, filed on Aug. 21, 2012.

(51) Int. Cl. A61K 49/18; G01N 33/574

(52) U.S. Cl. CPC A61K 49/1875; G01N 33/57496; G01N 2333/47

(57) ABSTRACT

An MRI contrast material includes tumor-targeting metal-loaded ferritin nanoparticles constructed with genetically modified ferritin coupled to a target-specific agent. Ferritin derived from Archaeoglobus flulgidus (AfFtn-AA) forms hollow nanocages surrounding paramagnetic or superparamagnetic metal core, storing a significantly greater quantity of iron (approximately 7,000 Fe ions per ferritin cage) or other paramagnetic or superparamagnetic metal than natural ferritins, and is conjugated via a short linker with a monoclonal antibody against a cell surface antigen overexpressed by a cancer, to selectively and efficiently attach to tumor cells to enhance MRI contrast. Significant T₂ contrast with diminished T₁ effect was observed owing to the heterogeneous nanoconjugate distribution when bound to cells. In a treatment method, after imaging, an external stimulus heats the cell-bound agent to release the metal and selectively destroy the targeted cells. The enhanced imaging and release of toxic metal ions provides simultaneous early detection and treatment.

### RELATED APPLICATIONS

The present application is a continuation of and claims the benefit of international application serial number PCT/US2013/055955 filed Aug. 21, 2013, which claims the benefit of U.S. provisional applications Ser. No. 61/691,346 filed Aug. 21, 2012, and Ser. No. 61/803,955 filed Mar. 21, 2013 entitled, “Ferritin-based tumor targeting agent, and imaging and treatment methods” by William Keun Chan Park, David R. Mills and Edward G. Walsh. The full text, including drawings and Appendices of those applications are hereby incorporated herein by reference. In addition, a Bibliography in this specification contains further technical detail regarding the procedures and materials described herein. For brevity, articles in the bibliography are referred to simply by (Author, year) in the disclosure below.

### GOVERNMENT SUPPORT

This invention was made with government support under grant number P20GM103421 awarded by the National Institute of General Medical Sciences of the National Institutes of Health. The government has certain rights in the invention.

### BACKGROUND

More than 571,950 people in the U.S. died from common cancers (colorectal, prostate, breast, lung and liver cancers), and more than 1.5 million new cancer cases were diagnosed in 2011 (American Cancer Society, 2011). Despite numerous technological and medical breakthroughs made in recent years, effective diagnosis and treatment of these cancers remain elusive. In order to overcome limitations regarding the lack of early detection methods and/or selective tumor-targeting therapeutic agents, current paradigms for cancer research continue to place an emphasis on discovery of improved tumor-specific biomarkers, on development of more sensitive detection/visualization methods for accurately assessing the location and extent of tumors, on treatment options and on selective delivery of anti-tumor agents to primary and secondary metastatic tumors.

Magnetic resonance imaging (MRI) is a versatile medical imaging modality capable of providing both structural and functional information using a variety of contrast weightings. For structural (conventional diagnostic) imaging, soft tissue contrast is produced by exploiting differences in T₁, T₂, or T₂* between different tissues (for example, between grey and white matter in the brain via T₁ weighting). Although many structures can be distinguished using endogenous contrast, it was found that some structures (such as tumors that have T₁ very similar to that of surrounding normal tissue) could be better visualized through the use of contrast agents. In X-ray imaging methods, contrast agents use high atomic number nuclei to increase attenuation and thereby reveal locations of contrast agent accumulation. In MRI, contrast agents are used to reduce T₁, T₂ or T₂* (or some combination of the three) to produce contrast in structures where the agent accumulates. The first approved MRI contrast agents were gadolinium chelates (e.g Gd-DTPA) which act as T₁ agents. Applications include brain tumor diagnosis: the contrast agent, normally restricted by the intact blood-brain barrier, passes out of the leaky vasculature of a malignant tumor and enters the interstitial space. T₁ weighted imaging 10-20 minutes post injection shows significantly increased signal intensity from the tumor owing to T₁ reduction whereas normal brain tissue with intact barrier does not show significant changes owing to the restriction of the contrast agent to the vascular compartment.

A second class of approved contrast agents has been developed around iron oxide (Fe₃O₄) nanoparticles. These superparamagnetic particles produce primarily T₂ and T₂* (susceptibility) contrast although some T₁ effect has been demonstrated. Since Fe₃O₄ is isoelectric at physiologic pH, a coating is required to maintain monodispersion. Dextran was the first coating used for an approved agent. Other coatings, such as polyethylene glycol (PEG) have been demonstrated useful for the purpose. An important characteristic for any contrast agent is the ability for detection at low concentrations. In this respect, the iron oxide particle agents demonstrate considerably higher relaxivity (defined as the change in relaxation rate per unit agent concentration) than those observed for the gadolinium chelates.

Recent efforts have examined the use of ferritin as a potential contrast agent (Uchida et al. 2006, 2008; Swift et al. 2009; Sana et al. 2010; Jordan et al. 2010). Ferritins are iron storage proteins that play a role in the maintenance of iron homeostasis. They function by converting soluble iron into a ferric complex (hydrite) that is stored in an internal cavity of the protein forming in essence, an iron nanocore (Chasteen and Harrison 1999; Harrison and Arosio 1996). Initial work involved the use of endogenous ferritin for estimation of iron concentrations in spleen, pancreas and liver as a means of assessing organ function using T₂ weighted image acquisitions. Natural ferritin complexes however, have been shown to have r1 and r2 values too low to act as effective contrast agents (Uchida et al. 2008 and our measurements reported in the discussion of Example 1, below). To improve the prospects of using ferritin as a basis for MRI contrast agents, modified forms have been developed with the aim of encapsulating more iron than in natural forms, with resultant improvements in relaxivity. One such form is the genetically engineered ferritin cage derived from Archaeoglobus fulgidus developed by Swift and Sana (Swift et al. 2009; Sana et al. 2010). This is a self-assembling spherical cage consisting of 24 subunits which is capable of storing on the order of 7000 Fe atoms per cage in a cavity approximately 8 nm in diameter with an overall hydrodynamic diameter of 14 nm for the entire complex. Advantages of using this complex include a very narrow distribution of particle size (Yoshimura 2006), relaxation enhancement through protein-associated water molecules (Rime et al. 2002), and biocompatibility and stability in biological systems (Mulder et al. 2006).

In previous studies, we developed a spontaneous transformation model for rat bile duct epithelial cells (BDEC) that culminated at high passage (p>85) in anchorage independent growth for cells plated on soft agar, and tumorigenicity when injected into immune deficient mice (Rozich et al. 2010). Briefly, by mid-passage (p31-85), BDEC showed alterations in morphology, onset of aneuploidy, increased growth rate with growth factor independence, decreased cell adhesion and loss of cholangiocyte markers expressed at low passage (p<30). We have recently developed an in vitro model of spontaneous transformation for rat prostate epithelial cells (PEC) that closely recapitulates many of the molecular and cellular changes observed during spontaneous transformation of rat BDEC. The rat prostate cells were isolated from dorso-lateral prostate lobes from mature Fisher 344 rats without prior carcinogen treatment as described previously (Britt et al. 2004; Mills et al. 2012-Exp Mol Path, in press). The development and characterization of the transformed rat PEC line used in the examples herein will be more fully described elsewhere in a forthcoming publication (Mills et al., manuscript in preparation).

However, as relevant to the present invention, previous studies in our laboratory have demonstrated that the transformed rat PEC used in this study express high levels of the cell adhesion protein, Necl-5, a cell surface glycoprotein that has been shown to promote cellular proliferation, migration and invasion of transformed cell lines (Sloan et al. 2004; Sato et al. 2004; Ikeda et al. 2004). While Necl-5 is barely detectable in normal epithelial cells, it is dramatically upregulated in many carcinomas including prostate, colorectal, lung, hepatocellular carcinoma (HCC) and other epithelial cancers (Faris et al. 1990; Chadeneau et al. 1991; Gromeier et al. 2000; Masson et al. 2001). The constitutive over-expression of Necl-5 in the rat PEC cell line makes it an attractive target for the development of future cancer detection and therapeutic strategies targeting CD155 or other human cancer markers.

### SUMMARY

In a first embodiment of the invention, a contrast agent for enhanced imaging, comprises metal-loaded, e.g., iron- or manganese-loaded synthetic ferritin nanoparticles coupled with a targeting agent, for example conjugated to an antibody, wherein the antibody or agent targets specific cells, e.g., tumor cells of a known type. Targeting involves binding to a receptor or surface molecule that is up-regulated in the cells, such that the contrast agent specifically or preferentially and effectively adheres to the cells and accumulates at the tumor; the MRI response of the metal-loaded ferritin provides enhanced imaging of the tumor. By providing a tissue-specific change in magnetic response properties, MRI imaging thus amounts to identifying or diagnosing tumor tissue in a subject or in an in vitro culture. In an exemplary imaging method using the contrast agent, antibody-linked iron-loaded ferritin material is administered to a subject or applied to a cell culture before imaging to enhance MRI imaging of the cells. When administered to a subject, either systemically or by local injection to a tumor site, the method may further include the step of confirming and/or quantifying the ferritin accumulation at the tumor (as evidenced, for example, by reduced T₂ and T₂* as compared to a baseline scan), and/or may further include the step of applying an externally-applied stimulus, such as a suitable magnetic field, in a region of the tumor, to locally elevate the temperature and/or promote release of toxic iron from the ferritin, thereby effectively and selectively treating or killing the tumor cells. The magnetic field may be of a strength and be reversed at a frequency effective to promote hyperthermia from energy absorption and Neel relaxation in the iron core nanoparticles. Alternatively, or in addition, an external magnetic field or other stimulus may be applied in a manner to cause the localized release of ionic iron held in the ferritin cage. The ferritin material is preferably an engineered material with a high capacity for holding iron, and may be further engineered to possess one or more large-dimension pores to enable enhanced release of iron therefrom, e.g., to increase the rate of release as a function of temperature or other stimulation or to initiate release at a high rate upon a relatively modest elevation of temperature. This aspect of the invention also contemplates external stimulation by non-magnetic means, such as by focused ultrasound, to promote the release of iron at the target tissue.

### BRIEF DESCRIPTION OF THE DRAWINGS

These and other features of the invention will be understood from the description and claims hereof, taken together with the Drawings, wherein:

FIG. 1 schematically illustrates preparation of the ferritin/mAb Necl-5 nanoconjugate ferritin material used in the examples herein;

FIG. 2 is a composite indirect immunofluorescence and transmission electron micrograph image confirming tumor targeting activity of the nanoconjugate material of FIG. 1;

FIG. 3A and FIG. 3B show MRI images of uniformly distributed conventional ferritin and the imaging ferritin of this invention, respectively, at various concentrations;

FIG. 4A and FIG. 4B show MRI images of the ferritin attached to tumor cells confirming attachment and enhanced imaging properties;

FIG. 5 shows iron assay results quantifying conjugates/cell data for the imaged sample; and

FIG. 6 shows signal contrast and intensity values.

### DETAILED DESCRIPTION

The invention will be understood from the following description of an exemplary embodiment and measurement results obtained therewith, together with discussion of the observed binding, magnetic and imaging characteristics reported below and their use in imaging, diagnosing and treating tissue conditions such as cancer. Briefly, the invention provides a new MRI contrast agent, namely cell-targeting ferritin cage nanoparticles loaded with iron or other magnetic or paramagnetic metal. The invention also provides diagnostic and treatment methods using the contrast agent.

Initially we describe in detail the preparation of an iron-loaded, cancer-targeting ferritin nanoparticle contrast agent and its properties.

### Methods and Materials

### Ferritin:

The ferritin used in the present study is a genetically engineered ferritin obtained from Archaeoglobus fulgidus. Cloning, expression and purification were performed following the methods previously described in Sana et al. (2010). Enrichment of the ferritin with iron (III) ions and the analysis of iron loading were achieved by following the methods reported in Liu et al. 2003; Glahn et al. 1995; and Bonomi and Pagani 1986. The process was repeated three times, and the average value for the number of iron (III) ion per each ferritin was determined to be 6,700. It was observed that iron loading beyond 7000 Fe/cage resulted in some difficulty in maintaining monodispersion in suspension, with precipitation possible due to aggregation.

### Conjugation of Iron-Enriched Ferritin and Anti-Necl-5 Monoclonal Antibody (MAb Necl-5):

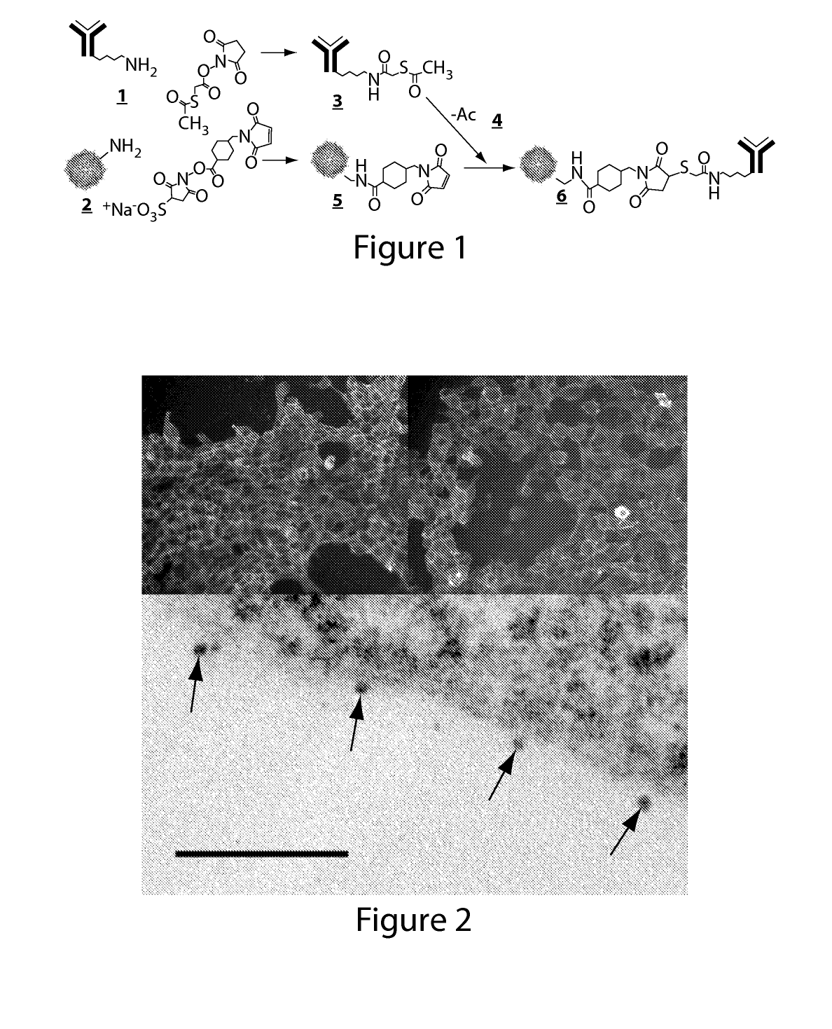

Generation and characterization of the Necl-5 specific mouse IgG monoclonal antibody (MAb 324.5) has been described previously (Hixson et al. 1986; Faris et al. 1990; Lim et al. 1996). To prepare the contrast agent, the two components, mAb Necl-5 and Fe(III)-enriched ferritin, were tethered by a convergent method. FIG. 1 schematically illustrates preparation of the ferritin/mAb Necl-5 nanoconjugate ferritin material 6 used in the examples herein. This diagram also illustrates the relative length and size of the components, ferritin (about 12.5 nm in diameter), antibody (about 10 nm in length) and the linker (about 1 nm) shown in the schema 1-6 of FIG. 1. Briefly, lysine residues of 1, mAb(Necl-5) were reacted with SATA (N-Succinimidyl S-Acetylthioacetate, 4 equiv. ThermoScientific) in a HEPES buffer solution (pH 7.5), which result in 3, a thioacetyl acetamide elongation. Separately, lysine residues of 2, Fe(III)-enriched ferritin were treated with Sulfo-SMCC (succinimidyl 4-[N-maleimidomethyl]cyclohexane-1-carboxylate, 4 equiv. ThermoScientific) in the same buffer solution to yield 5, which in turn, reacted with 4, the deacetylated thiol form of 3. The reaction of 4 with 5 proceeded in the presence of EDTA in order to suppress the disulfide formation between two 4 molecules. The desired conjugate 6 was obtained and isolated by a size exclusion chromatography (SEC) Superdex 200 10/300 GL column (GE Healthcare, Buckinghamshire, UK).

### In Vitro Preparation of Cells:

Transformed rat PEC were maintained in a 1:1 mixture of RPMI 1640 (Gibco, Carlsbad, Calif.) and MCDB 153 (Sigma-Aldrich, St. Louis, Mo.) supplemented with sodium bicarbonate (1.9 g/L), sodium pyruvate (0.5%), fetal bovine serum (FBS) (5%, Hyclone, Logan, Utah), epidermal growth factor (0.02 μg/ml, BD Biosciences, San Jose, Calif.), bovine pituitary extract (5 μg/ml, BD Biosciences), dexamethasone (2 mM in 95% EtOH), glutamine (1%), gentamycin (0.1 mg/ml, Gibco), ITS (0.25%, BD Biosciences), forskolin (2.5 μg/ml, Calbiochem, San Diego, Calif.) and Normocin and incubated at 37° C. in a 5% CO₂ humidified atmosphere. Cells were grown to approximately 75-80% confluence, and were trypsinized and washed in Hanks Balanced Salt Solution (HBSS; Sigma-Aldrich). Cell suspensions were incubated in the presence or absence of Necl-5 nanoconjugate in 1×PBS supplemented with 0.5% BSA at 4° C. for 1 hr with gentle rotation. Following two 5 min washes in HBSS, cells suspensions were mixed 1:1 with 1% SeaPlaque low melting temperature agarose (Lonza, Rockland, Me.) in 2 ml conical vials for subsequent imaging. Cell preparations in 2 ml vials (along with an undosed control cell sample) were scanned using the same procedure as for the uniform dispersion gel samples with relaxation rates and relaxivities calculated in the same manner. Each cell pellet contained approximately 2×10⁷ cells.

### Example 1

A targeted nanoconjugate version of the ferritin construct was prepared for in vitro testing as shown in FIG. 1 by binding a monoclonal antibody targeting the Necl-5 glycoprotein, expressed by many epithelial carcinomas. Transformed rat prostate epithelial cells (2.0×10⁷ cells per sample) were incubated with the targeted form of the ferritin nanoconjugate at three dose levels: 50, 100, and 200 μg conjugate per ml. After the incubation (37° C., 45 minutes), the samples were washed and centrifuged for three cycles. All of the washes including unbound conjugates were collected and analyzed for iron content using the bathophenanthroline disulfonic acid/sodium dithionate method described earlier (Bonomi and Pagani 1986).

### Magnetic Resonance Assessment of Relaxivity

For MR relaxivity measurements, iron loaded ferritin cages loaded to 6700 Fe/cage were uniformly dispersed in 1% agarose gel at concentrations of 1, 2, 5, 10, 20, 50, 100, 200, 500 and 1000 nM. Corresponding phantoms were prepared using natural horse ferritin. The gels were contained in 1.5 ml vials for scanning. Scans were acquired using a 3 Tesla Siemens Tim Trio system. A 32-channel head resonator was used for signal receive. Field shimming to second order was performed prior to acquisition of mapping scans. The ferritin vials, along with controls (agarose gel alone) were placed horizontally in a holder within the head resonator. Tomographic images 2 mm thick were acquired of the vials in cross-section with an in-plane resolution of 0.4 mm. For estimation of T₂ a multi-spin echo sequence was used with a repetition time of 1500 ms and 12 echo times ranging from 10 ms to 120 ms in 10 ms steps. In addition, gradient echo images were acquired to give an indication of susceptibility contrast (TR=1500 ms, TE=4-24 ms, six echoes). Inversion recovery was used for estimation of T₁ with a repetition time of 4000 ms and 12 inversion times ranging from 100 ms to 2400 ms. Relaxation time maps were formed by fitting signal intensity vs echo time (or inversion time) to the relevant signal equations using three-parameter nonlinear least squares fitting routines (Matlab). Relaxivity was determined using a linear fit for relaxation rate vs ferritin concentration.

### Results and Discussion

FIG. 2 is a composite indirect immunofluorescence and electron micrograph image showing the tumor targeting activity and imageability of the nanoconjugate material 6 of FIG. 1. Indirect immunofluorescence imaging demonstrated strong reactivity of the ferritin/mAb Necl-5 nanoconjugate (FIG. 2, top left) against transformed Necl-5 positive rat prostate epithelial cells that was comparable to anti-Necl-5 antibody alone (FIG. 2, top right). Furthermore, transmission electron microscopy (TEM) showed that the nanoconjugate binds to the rat prostate epithelial cells in a manner comparable to gold conjugated anti-Necl-5 antibody (FIG. 2, lower panel). These in vitro studies indicate that conjugation of the modified ferritin cage to anti-Necl-5 antibody did not affect the targeting specificity or reactivity of the antibody against the Necl-5 antigen.

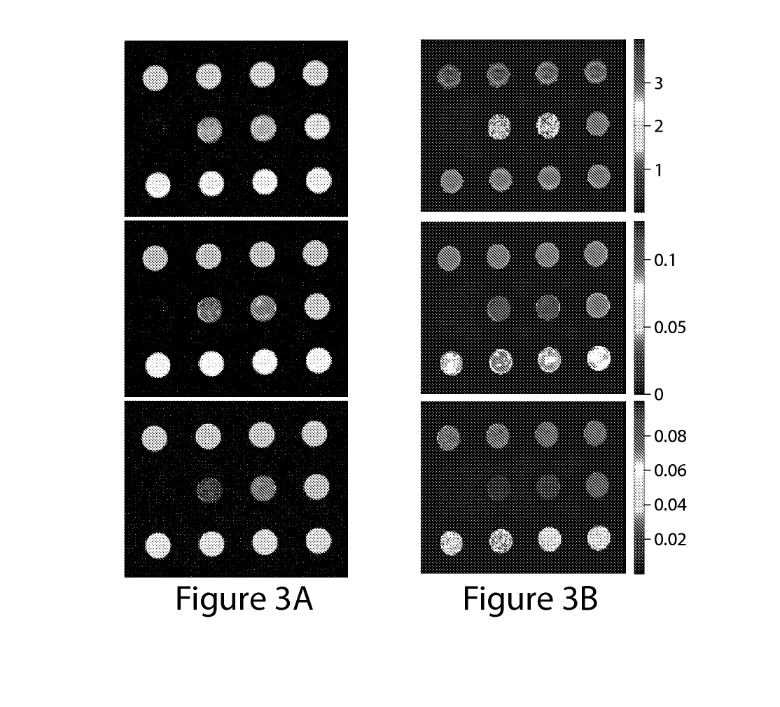

MRI imaging of phantoms made evident that contrast effects of all three weightings (T₁, T₂, and T₂*) were visible when the ferritins were evenly distributed in an agarose gel (FIG. 3A). FIG. 3A shows cross section images of the uniformly distributed ferritin (7000 FE/cage) samples, wherein the Top shows inversion recovery with inversion time=1200 ms, the Middle shows spin echo image with TE=20 ms, and the Bottom shows gradient echo image with TE=16 ms. FIG. 3B shows corresponding relaxation time maps for T₁ (top), T₂ (middle) and T₂* (bottom). Color scales on the right side are relaxation time in seconds. In all of the frames, the top row is the horse ferritin samples at concentrations of 1, 10, 100, and 1000 nM (left to right); the middle row consists of the engineered ferritin samples at the same concentrations and the bottom row contains gel only without ferritin. For the T₂ and T₂* weightings, contrast is evident at the shortest echo times (10 ms and 4 ms, respectively). The horse ferritin, which is here taken as indicative of endogenous ferritin or a conventional natural ferritin, did not show any significant contrast in the images, although a slight effect was noted in the T₂ and T₂* maps (FIG. 3B) while T₁ effect is negligible. Relaxivity (r₁, r₂) was calculated as the slope of the line resulting from a linear fit of relaxation rate vs concentration. The values for the ferritin loaded to 6700 Fe/cage were r₁=1290 mM⁻¹s⁻¹ and r₂=5742 mM⁻¹s⁻¹. These values were significantly higher than those obtained from the horse ferritin (r₁=0.674 mM⁻¹s⁻¹, n=95.54 mM⁻¹s⁻¹). This result compares favorably to commercial superparamagnetic iron oxide nanoparticle (SPION) imaging preparations as well as micelle-contained FePt variants (Taylor et al. 2011).

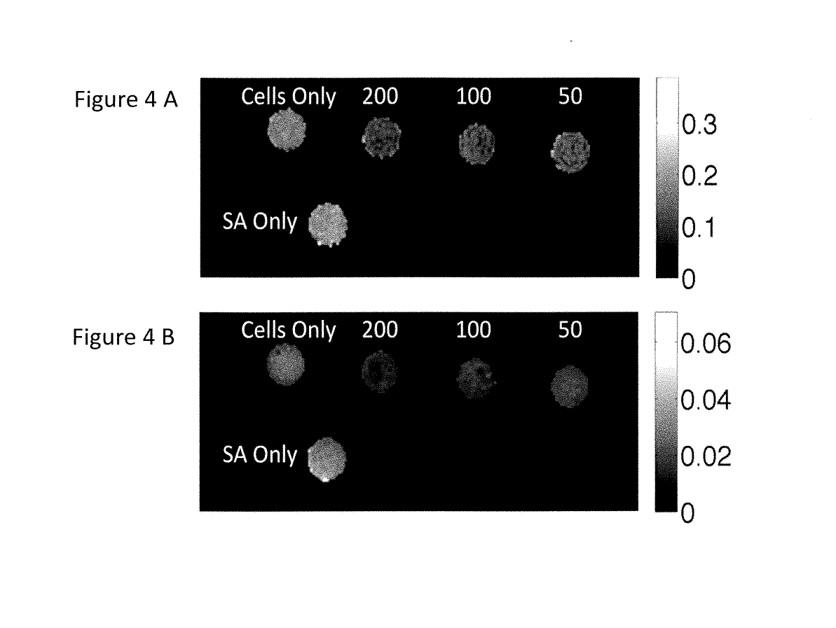

FIGS. 4A and 4B show relaxation time maps of tissue bound nanoconjugate. FIG. 4A illustrates T₂ and FIG. 4B illustrates T₂* relaxation time maps of the nanoconjugate when bound to target rat prostate epithelial cells. It was observed that the T₁ effect was negligible, whereas in the uniformly distributed case (FIG. 3B) the T₁ effect was clearly seen. This may relate to the heterogeneous particle distribution resulting in static dephasing (Bowen et al. 2002). Mean relaxation time values were determined for regions of interest taken from the center 80 pixels of the in vitro sample images, and are shown in TABLE 1. The entries are mean±standard deviation of relaxation times for the in vitro study. Circular regions of interest (100 pixels) were taken from the center of the vials. SA denotes soft agar.

TABLE 1

| Sample | T₂ (ms) | T₂ * (ms) |
|---|---|---|
| Cells Only | 172.1 ± 19.67 | 29.54 ± 1.811 |
| 50 μg/ml | 138.7 ± 21.54 | 20.52 ± 1.692 |
| 100 μg/ml | 111.2 ± 18.44 | 16.24 ± 1.981 |
| 200 μg/ml | 100.7 ± 18.24 | 11.73 ± 1.888 |
| SA Only | 198.7 ± 21.68 | 32.32 ± 2.312 |

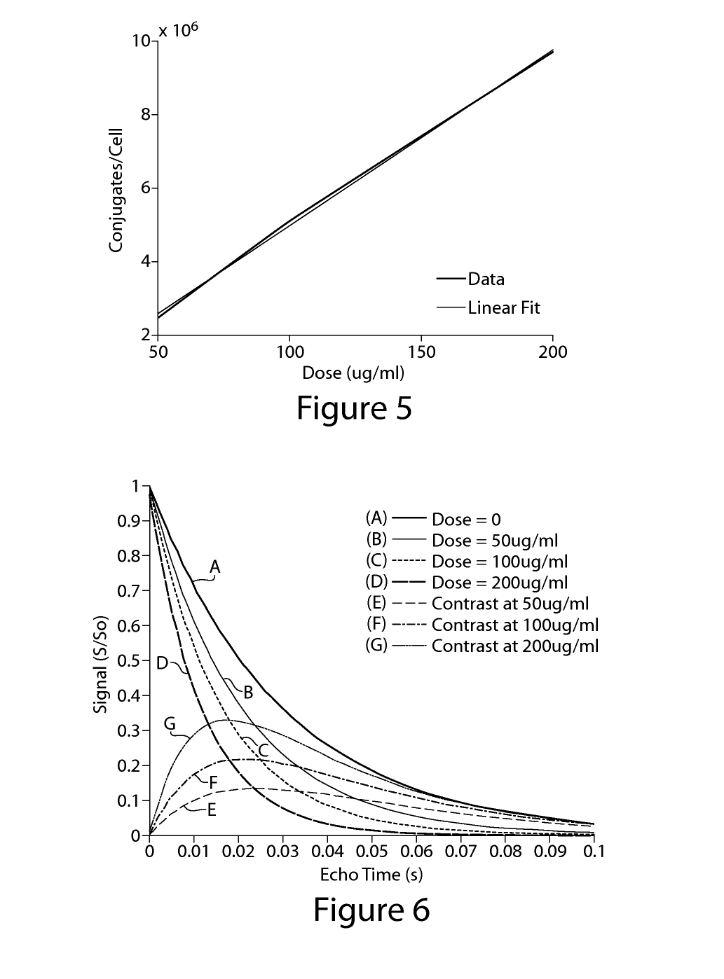

FIG. 5 is a plot of the conjugate retention vs dose for the in vitro preparation. The clear linear dependence indicates that receptor saturation was not reached even at the highest dose, and that greater binding is possible for this preparation with doses beyond 200 μg/ml. Assay results for the in-vitro preparation of Iron per cell are shown in TABLE 2. The iron concentrations were estimated based on the volume of the cell pellets, number of cells per pellet and quantity of iron per cell.

For the T₂* values determined in TABLE I, FIG. 6 shows the corresponding signal intensity and contrast curves to illustrate the optimum echo times based on the doses. Contrast is defined as the difference between the signal intensity curve at each concentration subtracted from the control. It was observed that as the dose level increases (and T₂*) decreases, that peak contrast increases, and the echo time corresponding to peak contrast decreases. The echo times for peak contrast vs. the control occur at 25 ms (50 μg/ml), 23 ms (100 μg/ml) and 19 ms (200 μg/ml). These magnitudes imply that for an image (pixel) signal-to-noise ratio of 20 in the baseline image, the contrast change will be detectable with a dose of 20 μg/ml for the in vitro preparation described above. That dose would correspond to approximately 0.62 μg/cell iron loading.

TABLE 2

| Dose (μg/ml) | Conjugates/Cell | Iron/Cell (pg) | [Fe] (nMol) |
|---|---|---|---|
| 50 | 2.5 × 10⁶ | 1.55 | 103.8 |
| 100 | 5.1 × 10⁶ | 3.17 | 211.8 |
| 200 | 9.7 × 10⁶ | 6.01 | 402.7 |

The high ratio of R₂*/R₂ is indicative of static dephasing (Bowen et al. 2002) resulting from local accumulations of particles as opposed to uniform distribution. Dependence of T₁ and T₂ in the presence of superparamagnetic nanoparticles has been described for uniform distribution using modified forms of the Solomon-Bloembergen-Morgan equations (Koenig et al. 1995; Bulte et al. 1999). These calculations predicted superparamagnetic particles as having a much smaller effect on T₁ than on T₂ owing to the large magnetic moment. This observation was confirmed in the uniform distribution measurements and may be the result of diffusion of associated water molecules through the ferritin channels (Aime et al. 2002). With respect to R₂ and R2*, compartmentalization causes the assumptions behind the quantum solution to fail, an effect previously described in cell-based studies (Weissleder et al. 1997; Majmudar et al. 1989). Compartmentalization is also accompanied by a substantial increase in the ratio R₂*/R₂ which is not predicted by the quantum solution. The quantum solution assumes the extreme motional narrowing condition, in which water diffusion between superparamagnetic particles is occurring on a time scale significantly shorter than the peak frequency offset and identical values for R₂ and R₂* are predicted. Compartmentalization of the particles results in bulk susceptibility producing local field inhomogeneities that render the assumption invalid. Monte Carlo simulations of water diffusing through local dipolar fields however, have been successfully employed in predicting the relationship between R₂ and R₂* for the case of particle compartmentalization (Weisskoff et al. 1994; Muller et al. 1991; Hardy and Hendelman 1989; Fisel et al. 1991; Majmudar and Gore 1988).

Changes in T₂ and T₂* were clearly distinguished in the in vitro preparation at a concentration (in the cell pellet) of 103 nMol. The minimum detectable concentration for the agent depends on a number of factors including cell density, magnetic field shim conditions in the region of the tissue binding the agent, the scan type (spin vs gradient echo) and scan parameters (repetition time, echo time, and geometric factors affecting signal to noise ratio). As seen from the binding assay (FIG. 5, TABLE 2) it appears likely that concentrations in excess of 400 nM can be produced in this in vitro preparation or an in vivo case with similar cell density, which would thus result in a very substantial contrast effect.

The foregoing experimental results establish the effective targeting and imaging of a specific protein by a ferritin construct, and quantification of the relevant MRI imaging and dosing parameters in an in vitro experimental model. In the study reported by Sana et al. (2010), a clear T₁ effect was observed at a field strength of 3 Tesla, the same field strength used in this study. This was verified in examples herein with the preparation in which ferritin particles were uniformly distributed in agarose gel. The lack of T₁ effect in the in vitro experiment may be the result of a reduced ability for free water to access the channels of the bound ferritin. If this is the case, use of the modified ferritin as a T₁ agent appears to be restricted to cases where the particles are maintained in an unbound state such that free water access to the ferritin channels is maximized. One example would be application as a blood pool agent for angiography studies where passage out of the microvasculature into the interstitial space is not desired. In such an application, a targeting ligand would not be required.

### Example 2

Rat high passage PEC (p93) cells and soft agar infiltrating (SAI)-selected prostate epithelial cells (PEC) were tumorigenic when injected into immunodeficient beige/nude mice. Tumor size was evaluated at four weeks post-injection for the high passage cells, and three weeks for the SAI-derived PEC tumors. SAI-derived tumors showed a shorter latency period than high passage derived tumors, and the average weight of removed tumors at the time of sacrifice was 0.2 grams (n=3, 4 weeks) and 0.76 grams (n=5, 3 weeks), for high pass and SAI injected cells, respectively. Indirect immunofluorescence imaging and western blotting each demonstrated that high passage (p102) and SAI-selected rat PRC expressed high levels of the cell surface glycoprotein Necl-5. To evaluate the ferritin-based contrast agent, in vivo MRI imaging of immunodeficient mice previously injected with PEC SAI cells was performed at 4 and at 24 hours after injection of anti-Necl-5/ferritin or ferritin alone, and was compared to baseline values taken before the ferritin injections. The nanoconjugate targeted tumor showed significant reduction of T₂ signal at 4 hours post-injection, and a substantially lesser reduction of T₂ at 24 hours, while the control, and regions of muscle tissue in both sets of mice were not substantially affected by either the targeted or the non-targeting ferritin.

Example 2 thus extends the results to in vivo application of an anti-Necl-5/ferritin nanoconjugate for imaging rat prostate epithelial cell tumors, and shows a time-dependent but dramatic difference in MRI response and imaging characteristics. Methods of imaging therefore advantageously include or are preceded by a preliminary time series dose/response sequence of measurements to acquire MRI characteristic data to optimize the interval between administration of the agent and imaging of the tumor.

### Example 3

In accordance with a further aspect of the invention the metal-filled ferritin cages, once bound to the target tissue, are caused to release the paramagnetic or superparamagnetic metal contents from their core. This process may be initiated or accelerated by heating, for example by applying a quickly-alternating magnetic field to generate heat, or by applying focused ultrasound to heat the particles and open pores of the ferritin cages. The high valence metal ions thus released from the core of the ferritin cages result in a locally toxic concentration of metal ions. Thus, imaging allows the treating physician to coordinate the excitation of the tumor-bound agent and release of the ferritin-caged metal to treat the tumor. The enhanced imaging characteristics enable earlier detection than would otherwise be possible, increasing the effectiveness of such a localized toxic treatment.

### General Considerations

The development of targeted imaging contrast agents with high specificity is an important step in the advancement of cancer diagnostics. Yet the diagnostic indicators for some cancers are relatively non-specific. For example, prostate cancer diagnosis relies on the use of prostate specific antigen (PSA) as a prostate tumor marker that has also served as a target for functionalized nanoparticle detection studies (Taylor et al. 2011). However, it was recently found that benign prostatic hyperplasia (BPH) also produces PSA, so that basing a diagnosis on PSA results in over-diagnosis and leads to unnecessary treatment (Chou et al. 2011). In accordance with the present invention, by targeting CD155, the human homologue of rat Necl-5, this diagnostic ambiguity would be eliminated. In examples herein we have demonstrated targeting of a ferritin-based metal complex to Necl-5 in a transformed rat prostate epithelial cell line model. A clear effect was seen for changes in T₂ and T₂* as would be reflected in spin echo and gradient echo imaging, respectively. The agent produced a visible effect (compared to a control) at a concentration of 102 nM Fe in the in vitro study along with an indication of the feasibility of binding to produce a concentration in excess of 400 nM. This is believed to be the first description of use of the modified ferritin complex as a contrast agent for targeting of a specific protein in an in vitro experimental model. As shown here, the in vitro data indicates that the modified ferritin conjugate has utility as both a T₂ and T₂* contrast agent when conjugated to an antibody of interest for targeting and imaging antigen-specific tissues. The antigen-specific tissues may be cancer cells or other diseased cells that express a specific cell surface molecule. Many such molecules have been characterized and associated with specific cancers or tissue pathologies; the antibody employed for targeting the ferritin nanoparticles may be an antibody to such a characterizing molecule, or may be an antibody to a relevant portion thereof.

In other embodiments, rather than the ferritin being conjugated to an antibody, equivalent specificity and effective accumulation and concentration at the relevant cells can be expected if the ferritin is clothed with the epitope, or active portion of the antibody responsible for binding. For example, the entire ferritin-epitope construct may be genetically engineered as a fusion protein. Furthermore, the targeted surface molecules may be a molecule that is specific to a highly invasive cell line, so that MRI images reveal specific information as to tumor type. Example 2 reports in vivo results imaging highly invasive tumors grown from soft agar infiltrating prostate epithelial cells. By specifically identifying surface markers and employing targeting antibodies for such cells, the techniques of the invention significantly advance early detection and treatment.

The magnitude of the relevant magnetic resonance parameters described above further indicates that other targeting functionalities—such as cloaking the ferritin in a targeting functionalized phospholipid or nanoemulsion as the delivery vehicle—can also be applied to advantage to achieve for in vivo delivery to tumor sites. A targeted nanoemulsion for in vivo use is compounded to allow the agent to circulate in the bloodstream sufficiently many times to accumulate specifically at the targeted tissue.

Once the relevant T₂ and T₂* values are determined, further baseline studies may be performed for a given targeting agent and target cell line to determine the optimum interval required after administering the ferritin nanoparticles for effective tissue binding to occur, so that diagnostic imaging and/or metal ion release therapy can be efficiently performed without taking multiple or comparative sets of before/after MRI scans. Comparison of pre- and post-administration MRI image data indicate tumorous regions of ferritin accumulation, and imaging protocols that display the difference will provide high contrast, tumor-specific imaging. For example, since the T₁ effect in EXAMPLE 1 was seen only when particles were uniformly suspended and unbound, so detection of a tumor would be revealed by T₂ and T₂* weighting. Once a baseline scan is acquired of the suspect region, tumor presence is revealed by reduction of T₂ and T₂* relative to the baseline scan when the contrast agent has been administered.

Coupling a tumor-targeting agent (e.g., an antibody) to the nanoparticle ferritin contrast agent in the present invention assures that the agent binds to the relevant tissue with high efficiency and specificity, so that while a dose/response relationship governs the image, only very small amounts of the contrast agent are needed for diagnostic imaging.

The foregoing describes a tissue-targeting nanoparticle MRI contrast agent and confirmatory measurements and observations that confirm its improved imaging characteristics, as well as its utility in methods of diagnosis and of treatment of specific diseased tissue or cancer conditions. The invention and illustrative methods being thus described, further variations and modifications will occur to those skilled in the art, and all such variations and modifications are understood to be within the scope of the invention and claims appended hereto.

### REFERENCES

Aime S, Frullano L, Crich S G, 2002. Compartmentalization of a gadolinium complex in the apoferritin cavity: a route to obtain high relaxivity contrast agents for magnetic resonance imaging. Angew. Chem. Int. Ed. 41:1017-1019.
American Cancer Society, 2011. Cancer facts and figures 2011. Atlanta Ga.
Bonomi F, Pagani S, 1986. Removal of ferritin-bound iron by DL-dihydrolipoate and DL-dihydrolipoamide. Eur J Biochem 155:295-300.
Bowen C V, Zhang X, Saab G, Gareau P J, Rutt B K, 2002. Application of the static dephasing regime theory to superparamagnetic iron-oxide loaded cells. Magn Reson Med 48:52-61.
Britt D E, Yang D F, Yang D Q, Flanagan D, Callanan H, Lim Y P, Lin S H, Hixson D C, 2004. Identification of a novel protein, LYRIC, localized to tight junctions of polarized epithelial cells. Experimental Cell Research 300:134-148.
Chadeneau C, Denis M G, Blottiere H M, Gregoire M, Douillard J Y, Meflah K, 1991. Characterization, isolation and amino terminal sequencing of a rat colon carcinoma-associated antigen. Int J Cancer 47:903-908.
Chasteen N D, Harrison P M, 1999. Mineralization in ferritin: an efficient means of iron storage. J Struct Biol 126:182-194.
Chou R, Croswell J M, Dana T, Bougatsos, Blazine I, Fu R, Gleitsmann K, Koenig H C, Lam C, Maltz A, Rugge J B, Lin K, 2011. Screening for prostate cancer—a review of the evidence for the U.S. preventive services task force. Ann Inter Med 155:762-771.
Erickson B M, Thompson N L, Hixon D C, 2006. Tightly regulated induction of the adhesion molecule necl-5/CD155 during rat liver regeneration and acute liver injury. Hepatology 43:325-334.
Faris R A, McEntire K D, Thompson N L, Hixson D C. 1990. Identification and characterization of a rat hepatic oncofetal membrane glycoprotein. Cancer Research 50:4755.
Fisel C R, Ackerman J L, Buxton R B, Garrido L, Belliveau J W, Rosen B R, Brady T J, 1991. MR contrast due to microscopically heterogeneous magnetic susceptibility: numerical simulations and applications to cerebral physiology. Magn Reson Med 17:336-347.
Glahn R P, Gangloff M B, van Campen D R, Miller D D, Wien E M, Norvell W A, 1995. Bathophenanthrolene disulfonic acid and sodium dithionite effectively remove surface-bound iron from caco-2 cell monolayers. J Nutr 125:1833-1840.
Gromeier M, Lachmann S, Rosenfeld M R, Gutin P H, Wimmer E. 2000. Intergeneric poliovirus recombinants for the treatment of malignant glioma. Proc Natl Acad Sci USA 97: 6803-6808.
Hardy P A, Henkelman R M, 1989. Transverse relaxation rate enhancement caused by magnetic particulates. Magn Reson Imaging 7:265-275.
Harrison P, Arosio P, 1996. The ferritins: molecular properties, iron storage function and cellular regulation. Biochem Biophys Acta Bioenergetics 1275:161-203.
Hixson D C, McEntire K, Chesner I, Faris R, Weltman J, Marceau N. 1986. Monoclonal antibody (MAb) recognizing a glycoprotein absent from normal tissues but present on transplantable (THC) and primary (PHC) hepatocellular carcinomas induced by azo dye. Proceedings of AACR 27:365.
Ikeda W, Kakunaga S, Takekuni K, Shingai T, Satoh K, et al. 2004. Nectin-like molecule-5/Tage4 enhances cell migration in an integrin-dependent, Nectin-3 independent manner. J Biol Chem 279: 18015-18025.
Jordan V C, Caplan M R, Bennett K M, 2010. Simplified synthesis and relaxometry of magnetoferritin for magnetic resonance imaging. J Magn Med 64:1260-1266.
Lim Y P, Fowler L C, Hixson D C, Wehbe T, Thompson N L. 1996. TuAg.1 is the liver isoform of the rat colon tumor-associated antigen pE4 and a member of the immunoglobulin-like supergene family. Cancer Research 56: 43034.
Liu X, Jin W, Theil E C, 2001. Opening protein pores with chaotropes enhances Fe reduction an dehelation of Fe from the ferritin biomaterial. Proc Natl Acad Sci USA 100:3653-3658.
Majmudar S, Zoghbi S S, Gore J C, 1989, The influence of pulse sequences on the relaxation effects of superparamagnetic iron oxide contrast agents. Magn Reson Med 10:289-301.
Majmudar S, Gore J C, 1988. Studies of diffusion in random fields produced by variations in susceptibility. J Magn Reson 78:41-55.
Masson D, Jarry A, Baury B, Blanchardie P, Laboisse C, et al. 2001. Overexpression of the CD155 gene in human colorectal carcinoma. Gut 49: 236-240.
Mulder W J M, Strijkers G J, van Tilborg G A F, Griffioen A W, Nicolay K, 2006. Lipid-based nanoparticles for contrast-enhanced MRI and molecular imaging. NMR Biomed 19:142-164.
Muller R N, Gillis P, Moiny F, Roch A, 1991. Transverse relaxivity of particulate MRI contrast media: from theories to experiments. Magn Reson Med 22:178-182.
Rozich R A, Mills D R, Brilliant K E, Callanan H M, Yang D Q, Tantravahi U, Hixson D C. 2010. Accumulation of neoplastic traits prior to spontaneous in vitro transformation of rat cholangiocytes determines susceptibility to activated ErbB-2/Neu. Experimental and Molecular Pathology 89: 248-259.
Sana B, Johnson E, Sheah K, Poh C L, Lim S, 2010. Iron-based ferritin nanocore as a contrast agent. Biointerphases 5(3):FA48-FA52.
Sana B, Poh C L, Lim S, 2011. A manganese-ferritin nanocomposite as an ultrasensitive T, contrast agent. Chem. Commun., 2012, 48, 862-864.
Sato T, Irie K, Ooshio T, Ikeda W, Takai Y. 2004. Involvement of heterophilic trans-interaction of Necl-5/Tage4/PVR/CD155 with nectin-3 in formation of nectin- and cadherin-based adherens junctions. Genes Cells 9: 791-799.
Sloan K E, Eustace B K, Stewart J K, Zehetmeier C, Torella C, et al. 2004. CD155/PVR plays a key role in cell motility during tumor cell invasion and migration. BMC Cancer 4: 73.
Swift J, Butts C A, Cheung-Liu J, Yerubandi V, Dmochowski, 2009. Efficient self-assembly of archaeoglobus fulgidus ferritin around metallic cores. Langmuir 25:5219-5225.
Taylor R M, Huber D L, Monson T C, Abdul-Medhi A, Bisoffe M, Sillerud L O, 2011. Multifunctional iron platinum stealth innumomicelles: targeted detection of human prostate cancer cells using both fluorescence and magnetic resonance imaging. J Nanopart Res 13:4717-4729.
Uchida M, Terashima M, Cunningham C H, Suzuki Y, Willits D A, Willis A F, Yang P C, Tsao P S, McConnell M V, Young M J, Douglas T, 2008. A human ferritin iron oxide nano-composite magnetic resonance contrast agent. Magn Reson Med 60:1073-1081.
Uchida M, Flenniken M L, Allen M, Willits D A, Crowley B E, Brumfield S, Willis A F, Jackiw L, Julita M, Young M J, Douglas T, 2006. Targeting of cancer cells with ferromagnetic ferritin cage nanoparticles. J Am Chem Soc 128:16626-16633.
Weisskoff R M, Zuo C S, Boxerman J L, Rosen B R, 1994. Microscopic susceptibility variation and transverse relaxation: theory and experiment. Magn Reson Med 31:601-610.
Weissleder R, Cheng H C, Bogdanova A, Bogdanov Jr A, 1997. Magnetically labeled cells can be detected by M R imaging. J Magn Reson Imaging 7:258-263.
Yoshimura H, 2006. Protein-assisted nanoparticle synthesis. Colloids Surf A 282-283:464-470.

1. A contrast agent for enhanced imaging, the agent comprising high-capacity ferritin nanoparticles loaded with metal ions and linked to an antibody or epitope thereof, wherein the antibody targets an antigen up-regulated in tumor cells, such that the contrast agent administered to a subject specifically and effectively accumulates at a tumor and the MRI response of the metal-loaded ferritin results in enhanced imaging of the tumor.

2. An imaging or treatment method comprising the steps of administering or applying to a subject or a cell culture, iron-loaded ferritin nanoparticles linked to targeting material, wherein the targeting material binds to a cell surface molecule that is up-regulated in tumor cells such that the nanoparticles selectively accumulate in the region of the tumor cells, the iron-loaded ferritin material having characteristic T₁, T₂ and/or T₂* response that is effective to enhance MRI imaging of the tumor cells.

3. The method of claim 2, further comprising the step of applying an external perturbation such as an alternating magnetic field in a region of the tumor to locally elevate temperature of iron held within or released by the ferritin thereby selectively killing the tumor cells.

4. The method of claim 2, further comprising the step of initiating release of cytotoxic iron ions from the ferritin to selectively kill the tumor cells.

5. The method of claim 4, wherein the step of initiating release comprises applying a magnetic exciting field to a region about the tumor such that magnetic field-induced hyperthermia promotes shedding of ionic iron from the ferritin.

6. The method of claim 4, wherein the ferritin is a synthetic ferritin forming a cage structure having one or more large pores to enhance shedding of iron held within in the ferritin, and wherein the magnetic field is adapted to promote one or more effects selected from among Neel relaxation heating and ultrasonic kinetic heating from Brownian-like motion.

7. The contrast agent of claim 1, wherein the ferritin is a synthetic ferritin forming a nanocage structure that holds over 2000, preferably over 5000 iron ions, and the targeting material is an antibody or epitope of an antibody linked to or conjugated to the ferritin.

8. The contrast agent of claim 7, wherein the contrast agent is produced as a recombinant fusion protein of ferritin and the epitope of an antibody, and is loaded with iron to form a monodisperse nanoparticle agent for administration to a subject.

9. The contrast agent claim 8, adapted to selectively enhance image contrast either bound to a target tissue or in a free fluid by application of two or more different imaging protocols.

10. The method of claim 2, wherein the ferritin is administered either systemically or locally injected directly to a tumor site.

11. The contrast agent of claim 1, adapted for treatment and diagnosis wherein the ferritin nanoparticles are engineered to release toxic iron upon external stimulation so as to selectively destroy targeted cells.

12. (canceled)

13. The agent of claim 11, wherein the external stimulus effects heating and/or vibration of the ferritin nanoparticle bound to cells.

14. The agent of claim 11, wherein the ferritin is engineered to self-assemble from multiple copies of a basic peptide into a nanocage structure that incorporates ionic iron, and to have pores open enabling enhanced release of the incorporated ionic iron for selective and localized tissue destruction.

15. A method of claim 2, further comprising the step of applying an external stimulus to release metal ions from the high capacity ferritin so as to attack and/or destroy targeted tumor cells to which the nanoparticles are bound.

16. (canceled)

17. The method of claim 15, wherein the ferritin has a capacity of more than about 500, preferably over 3000, over 5000 or about 7000 iron or paramagnetic metal ions, such as a synthetic ferritin derived from Archaeoglobus fulgidus or an exotic organism, modified to form a self-assembling cage having open pores, recombinant produced and wherein the targeting agent includes one or more of an antibody to a disease marker, or to a characteristic cell surface glycoprotein or other cell-related functional targeting agent.

18. The method of claim 15, comprising the step of MRI imaging to identify presence of the cells targeted by the targeting agent, and further comprising the step of applying energy to the imaged region to release cytotoxic ions from the nanoparticles to selectively destroy the cells, wherein the energy may include a rapidly oscillating magnetic field, ultrasound, and electric force field, and/or wherein the cytotoxic ions may include iron, of other paramagnetic metal ions or combinations thereof.

19. A method of detection or treatment of cancer, the method comprising:
coupling to ferritin nanoparticles an antibody or targeting agent that targets and binds to a marker of an invasive or resistant tumor cell of interest;
administering a formulation of the targeting agent/ferritin to a subject so that it selectively, preferentially or effectively binds to a marker of a type of invasive or resistant tumor cell of interest and changes MRI response characteristics of the invasive or resistant tumor cells of interest to thereby identify an invasive or resistant tumor, and
further comprising the step of exciting the nanoparticles bound to the tumor to selectively and locally treat the tumor.

20. (canceled)

21. The method of claim 19, wherein the targeting agent specifically targets cells of an aggressive or invasive tumor, whereby enhanced MRI imaging enables substantially simultaneous detection and treatment at an early stage or prior to substantial growth or metastasis of the tumor.

22. The method of claim 19, wherein the step of
exciting the ferritin nanoparticles is performed by externally stimulating the nanoparticles to release locally toxic metal to kill the tumor cells with or without performing an imaging step.

23. The contrast agent of claim 1, wherein the ferritin is a synthetic ferritin forming a nanocage structure that holds substantially more metal ions than a natural ferritin, such as over 2000, preferably over 5000 iron ions or hundreds of manganese ions, and the targeting material is an antibody or epitope of an antibody linked to or conjugated to the ferritin by a short linker such that the ferritin nanocage effectively binds to a targeted tissue, and the ferritin is adapted to locally release a toxic level of free metal ions when a rapidly alternating magnetic field is applied to the tissue.
